(19) United States
(12) Patent Application Publication
(10) Pub. No.: US 2005/0019745 A1
(43) Pub. Date: Jan. 27, 2005

(54) Random array of microspheres

(75) Inventors: Krishnan Chari, Fairport, NY (US); Zhanjun J. Gao, Rochester, NY (US); Joseph S. Sedita, Albion, NY (US); Ramasubramaniam Hanumanthu, Rochester, NY (US); Charles P. Lusignan, Rochester, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/625,428

(22) Filed: Jul. 23, 2003

(51) Int. Cl. C12Q 1/00

(52) U.S. Cl. 435/4

(57) ABSTRACT

A method for making an element containing an array of microspheres on a support, the method comprising the steps of: coating a support with a coating composition to form a receiving layer with a modifiable elastic modulus; coating on the receiving layer a dispersion of microspheres in a carrier fluid; modifying the modulus to allow the microspheres to partially submerge into the intermediate layer; removing the fluid medium from the suspension of microspheres; and fixing the microspheres on the receiving layer so that the element can withstand wet processing.

### CROSS REFERENCE TO RELATED APPLICATIONS

This application relates to commonly assigned copending application Ser. No. 09/942,241 (DN 82300), filed Aug. 29, 2001 entitled RANDOM ARRAY OF MICROSPHERES. The copending application is incorporated by reference herein for all that it contains.

### FIELD OF THE INVENTION

The present invention concerns biological or sensor microarray technology in general. In particular, it concerns a microarray coated on a substrate that contained no sites designated prior to coating to attract the microspheres.

### BACKGROUND OF THE INVENTION

Ever since it was invented in the early 1990s (Science, 251, 767-773, 1991), high-density arrays formed by spatially addressable synthesis of bioactive probes on a 2-dimensional solid support has greatly enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. Nos. 5,412,087, and 5,489,678 demonstrate the use of a photolithographic process for making peptide and DNA microarrays. The patent teaches the use of photolabile protecting groups to prepare peptide and DNA microarrays through successive cycles of deprotecting a defined spot on a 1 cm×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive. An ink jet approach is being used by others (e.g., U.S. Pat. Nos. 6,079,283; 6,083,762; and 6,094,966) to fabricate spatially addressable arrays, but this technique also suffers from high manufacturing cost in addition to the relatively large spot size of 40 to 100 μm. Because the number of bioactive probes to be placed on a single chip usually runs anywhere from 1000 to 100000 probes, the spatial addressing method is intrinsically expensive regardless how the chip is manufactured. An alternative approach to the spatially addressable method is the concept of using fluorescent dye-incorporated polymeric beads to produce biological multiplexed arrays. U.S. Pat. No 5,981,180 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information. U.S. Pat. No. 6,023,540 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach significantly expands the spectral barcoding capacity in micro spheres.

Even though the “spectrally addressed microsphere” approach does provide an advantage in terms of its simplicity over the old fashioned “spatially addressable” approach in microarray making, there are still needs in the art to make the manufacture of biological microarrays less difficult and less expensive.

U.S. Ser. No. 09/942,241 teaches various coating methods but exemplifies machine coating, whereby a support is coated with a fluid coating composition comprising microspheres dispersed in gelatin. Immediately after coating, the support is passed through a chill set chamber in the coating machine where the gelatin undergoes rapid gelation and the microspheres are immobilized.

While that invention provides a huge manufacturing advantage over then existing technologies, it needs some refinement in order to maximize its full potential value to the art. The problem is that during such machine coating and rapid gelation, the gelling agent tends to cover the surface of the microspheres, thereby preventing the analyte (such as DNA) from penetrating through the gel overcoat and hybridizing with probes on the surface of the microspheres.

U.S. Ser. No. 10/062,326, filed Jan. 31, 2002, overcomes the problem outlined above by enzymatically removing the gelling agent from the surface of the microspheres without damaging their integrity or the DNA probes on their surfaces. The enzyme treated surface maintains its physical integrity through the entire DNA hybridization process and the microarray shows a very strong hybridization signal.

The advantage of U.S. Ser. No. 10/062,326 is that enzyme digestion can be easily controlled to remove the required amount from the gel overcoat. Further, the enzyme, a protease, is readily available and economical to obtain. However, there is a disadvantage in that an additional process (enzyme digestion) is required and this involves additional time and cost.

U.S. Ser. No. 10/092,803, filed Mar. 7, 2002 describes a process of preparing a random bead micro-array by coating a suspension of micro-spheres without gelling agent but containing a cross-linker for the gelling agent onto a receiving layer capable of undergoing sol-gel transition. The micro-spheres partially submerge into the receiving layer that is then cross-linked to form a micro-array. While this approach is an improvement over U.S. Ser. No. 09/942,241, filed Aug. 29, 2001, it is not completely successful in preventing deposition of gelling agent onto the surfaces of the micro-spheres because the gelling agent in the receiving layer can dissolve in the aqueous suspension and re-deposit onto the micro-spheres when the suspension is spread on the receiving layer. Furthermore, the presence of cross-linker in the suspension can cross-link biological molecules on the surfaces of the micro-spheres and render them ineffective as probes.

A method is needed wherein a suspension of micro-spheres can be spread onto a receiving layer wherein the material of the receiving layer does not dissolve in the suspension or medium in which the micro-spheres are being transported. Furthermore, the composition of the receiving layer has to be such so as to permit sufficient submerging of the micro-spheres to prevent lateral aggregation when the solvent in the suspension is removed by evaporation.

### SUMMARY OF THE INVENTION

The present invention provides a method of making a microarray wherein the microspheres are randomly dispersed on a receiving layer and have surfaces exposed above the receiving layer that are free to interact with the analyte. The present invention discloses:

A method of making a microarray comprising the steps of:
providing a support;
coating on the support a receiving layer to receive microspheres, the receiving layer being capable of cross-linking to render it insoluble in the fluid carrying the micro-spheres;
cross-linking the receiving layer to achieve an elastic modulus to permit partial submerging of the micro-spheres;
coating on the cross-linked receiving layer a dispersion of microspheres in a carrier fluid;
allowing the microspheres to partially submerge into the receiving layer;
evaporating off the carrier fluid; and
allowing further cross-linking in the receiving layer to render the micro-array robust to wet processing.

In another embodiment of the invention, there is disclosed an element comprising randomly fixed microspheres, the element comprising:
a) a support; on which is disposed
b) a water-insoluble, crosslinked layer; and
(c) microspheres partially submerged in the crosslinked layer;
   wherein the microspheres, upon deposition on the layer, have surfaces exposed above the crosslinked layer, such exposed surfaces having probes attached that are capable of interacting with analytes applied to the element.

The receiving layer and the support are characterized by an absence of sites designed to specifically interact physically or chemically with the microspheres. Hence, the distribution of the microspheres is not predetermined or directed, but is entirely random.

The invention utilizes a unique coating technology to prepare a microarray on a substrate that need not be pre-etched with microwells or premarked in any way with sites to attract the microspheres, as disclosed in the art. By using unmarked substrates or substrates that need no pre-coating preparation, the present invention provides a huge manufacturing advantage compared to the existing technologies. The invention discloses a method whereby color addressable mixed beads in a dispersion are randomly distributed on a receiving layer that has no wells or sites to attract the microspheres.

The present invention provides a microarray that is less costly and easier to prepare than those previously disclosed because the substrate does not have to be modified; nevertheless the microspheres remain immobilized on the substrate.

Further, the present invention provides a microarray wherein, in contrast to U.S. Ser. No. 09/942,241, filed Aug. 29, 2001, the bead surfaces are exposed but without employing the additional process step (enzyme digestion) disclosed in U.S. Ser. No. 10/062,326, filed Jun. 3, 2002. Exposed bead surfaces facilitate easier access of the analyte to probes attached to the surfaces of the beads. By “analyte” is meant molecules with functionalities capable of interacting chemically or physically with specific moieties on the bead surface, herein called “probes”. In the present invention, the analyte is primarily nucleic acids or proteins.

One of the keys elements of the present invention is the selection of the receiving layer. The receiving layer must have a desired physical property that allows the microspheres to sufficiently submerge in the receiving layer thereby preventing lateral aggregation. Specific requirements on the physical properties of the receiving layer will be discussed in details later.

### BRIEF DESCRIPTION OF THE DRAWINGS

FIGS. 1a to 1e are schematics showing one method employed in the prior art for preparing a bead microarray. FIG. 1a shows any suitable support; FIG. 1b shows a fluid layer containing micro-spheres, gelling agent and a chemical cross-linking agent spread over the support of FIG. 1a; FIG. 1c shows the gelling agent undergoing sol-gel transition and thereby immobilizing the beads; FIG. 1d shows the evaporation of fluid from the coating composition to form the micro-array; and FIG. 1e shows the crosslinking reaction going to completion to permanently fix the beads in the array, leaving a film of gelling agent on the surface of the microsphere.

FIGS. 2a to 2g are schematics of another prior art process of preparing a random bead micro-array wherein FIG. 2a shows any suitable support; FIG. 2b shows the support coated with non-cross-linked gelling agent or precursor to a gelling agent; FIG. 2c shows a fluid carrying micro-spheres bearing probes and a cross-linker for the gelling agent disposed on the support of FIG. 2b; FIG. 2d shows the micro-spheres of FIG. 2c sinking into the layer with the gelling agent. The layer with the gelling agent undergoes sol-gel transition and thereby immobilizes the beads. FIG. 2e shows the evaporation of fluid from the coating composition; FIG. 2f shows the final micro-array; the beads still have a coating of polymer on their surfaces because of dissolution of gelling agent into the aqueous suspension.

FIGS. 3a to 3g are schematics of one embodiment of the present invention wherein FIG. 3a shows any suitable support; FIG. 3b shows a cross-linkable composition and chemical cross-linking agent spread over the support of FIG. 3a to form a receiving layer; FIG. 3c shows cross-linking of the material of the receiving layer the elastic modulus of which is adjusted to permit indentation by micro-spheres in a fluid suspension that will be spread over it; FIG. 3d shows a fluid suspension containing micro-spheres spread over the cross-linked receiving layer of FIG. 3c; FIG. 3e shows the micro-spheres partially sinking into the receiving layer; FIG. 3f shows the evaporation of fluid to expose the surfaces of the micro-spheres; FIG. 3g shows further chemical cross-linking of the receiving layer to make the micro-array robust to wet processing.

FIGS. 4a to 4g are schematics of another embodiment of the present invention wherein FIG. 4a shows any suitable support; FIG. 4b shows a cross-linkable composition spread over the support of FIG. 4a to form a receiving layer; FIG. 4c shows the receiving layer cross-linked by ultra-violet (UV) radiation, ionizing radiation or electron beam irradiation to an elastic modulus sufficient to permit indentation by micro-spheres in a fluid suspension that will be spread over it; FIG. 4d shows a fluid suspension containing micro-spheres spread over the cross-linked receiving layer of FIG. 4c; FIG. 4e shows the micro-spheres partially sinking into the receiving layer; FIG. 4f shows the evaporation of fluid to expose the surfaces of the micro-spheres; FIG. 4g shows further cross-linking of the receiving layer by UV radiation, ionizing radiation or electron beam irradiation to make the micro-array robust to wet processing.

FIG. 5 is yet another diagram of a process of our invention wherein FIG. 5a shows any suitable support; FIG. 5b shows a fluid containing a gelling agent and a slow acting chemical cross-linking agent for the gelling agent spread over the support of FIG. 5a to form a receiving layer; FIG. 5c shows the gelling agent in the receiving layer undergoing sol-gel transition to an elastic modulus sufficient to permit indentation by the micro-spheres; FIG. 5d shows a fluid suspension containing micro-spheres at a temperature below the sol-gel transition of the gelling agent in the receiving layer spread over the receiving layer of FIG. 5c; FIG. 5e shows the micro-spheres partially sinking into the receiving layer; FIG. 5f shows the evaporation of fluid to expose the surfaces of the micro-spheres; FIG. 5g shows chemical cross-linking of the receiving layer going to completion to make the micro-array robust to wet processing.

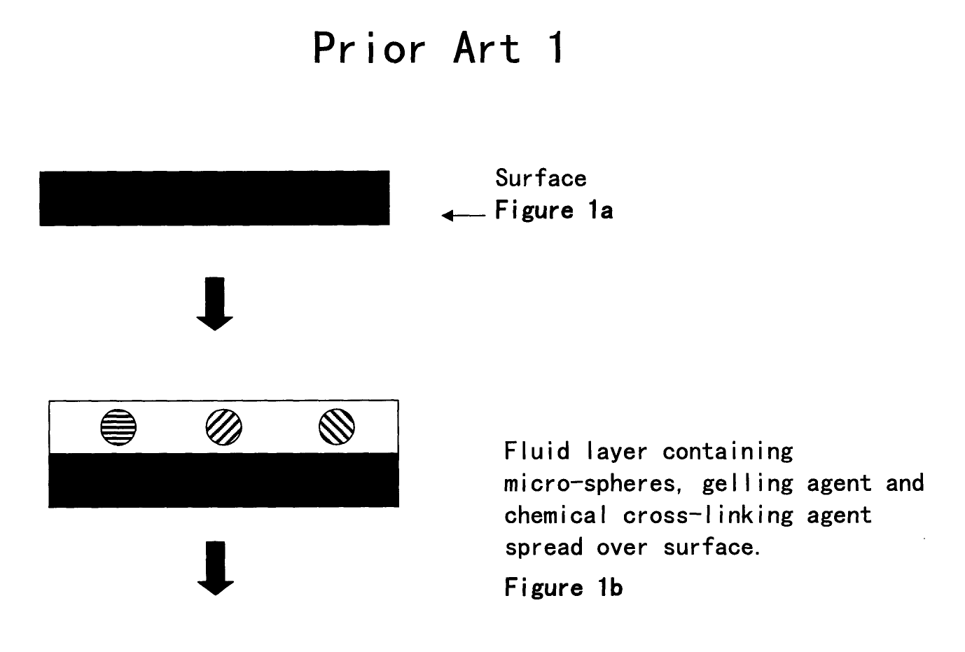

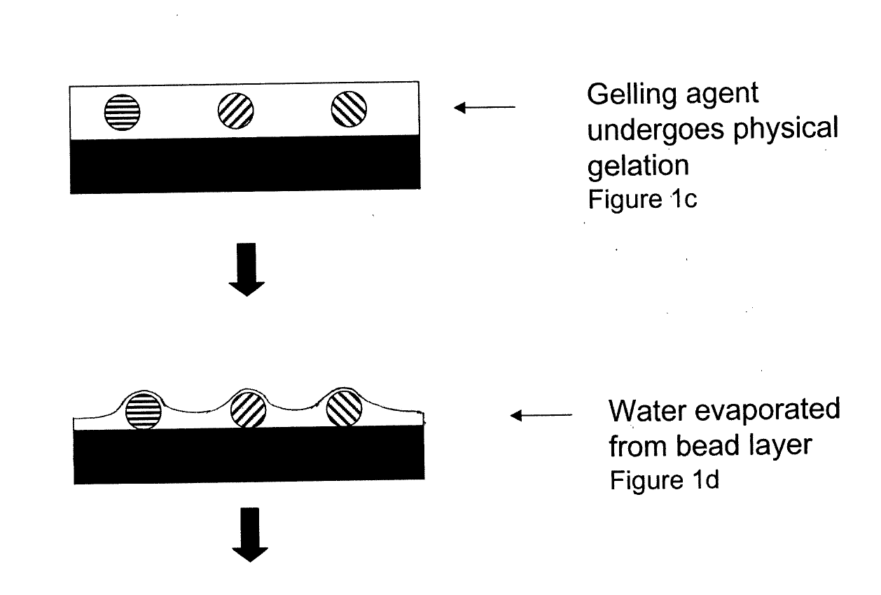

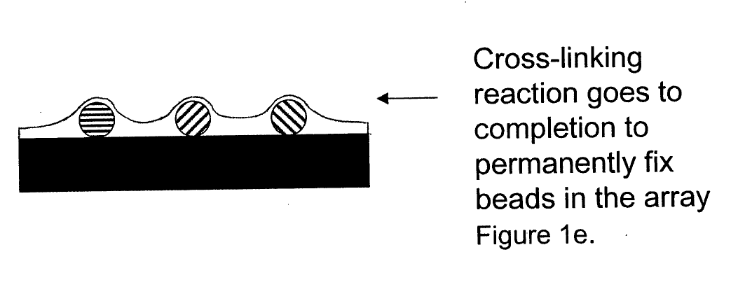

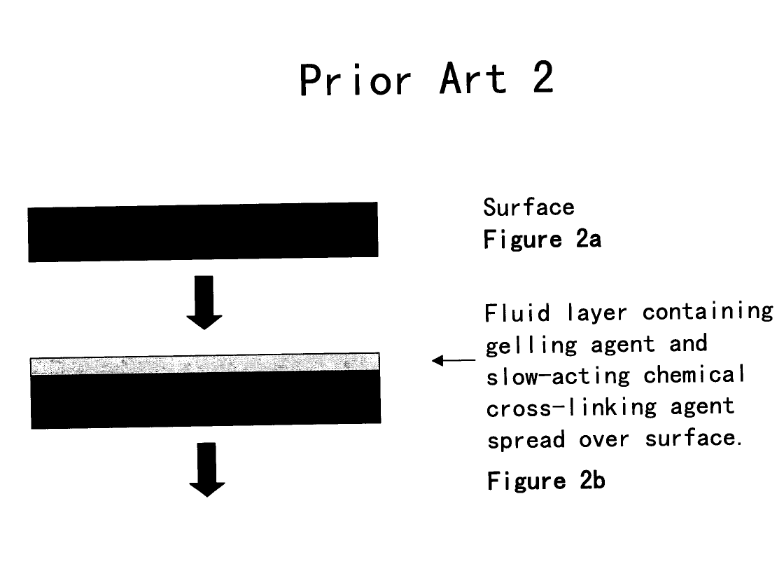

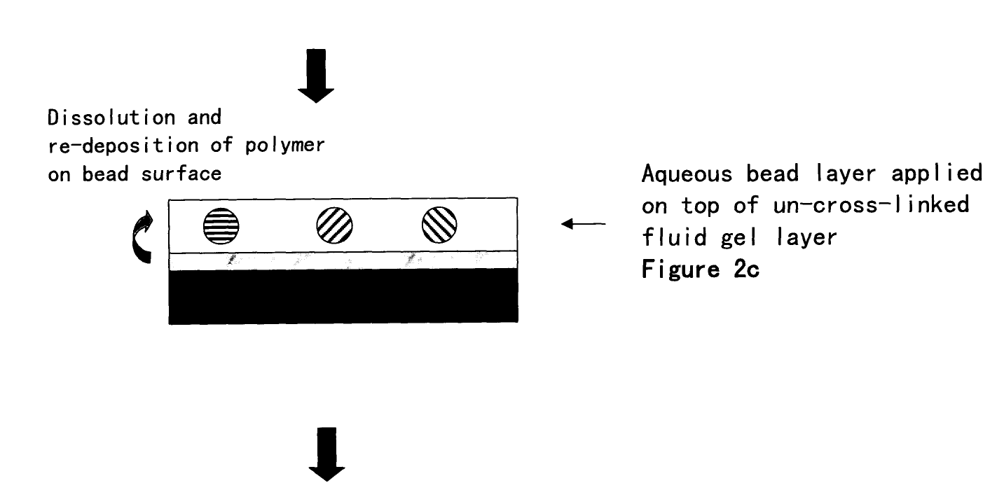

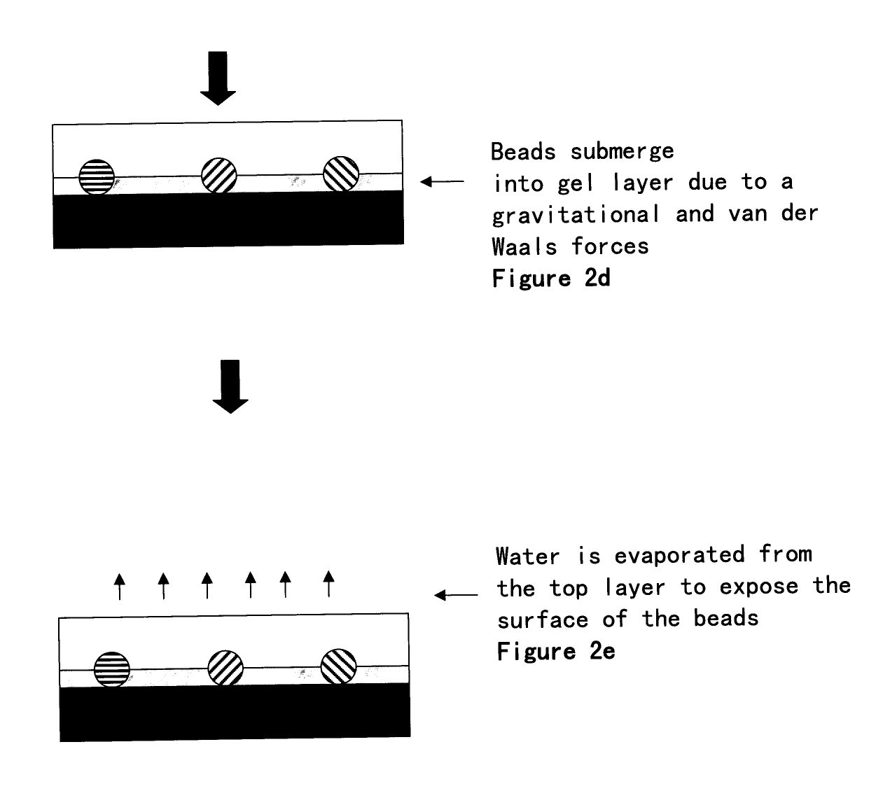

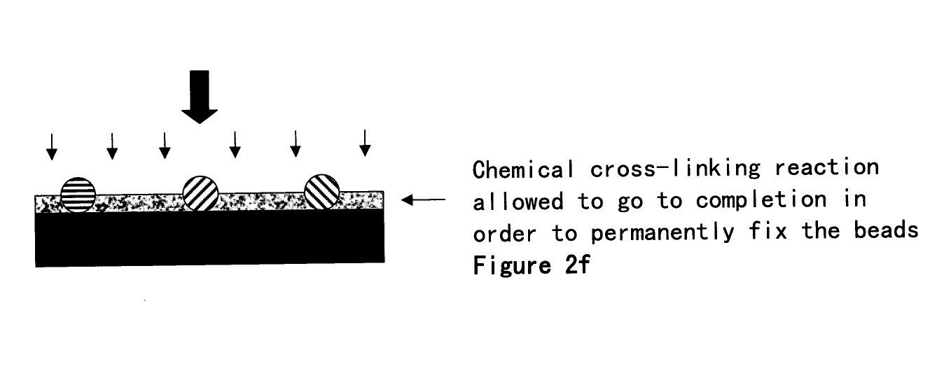

### DETAILED DESCRIPTION OF THE INVENTION

As used herein, the term “sol-to-gel transition” or “gelation” means a process by which fluid solutions or suspensions of particles form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation that is of the latter type. The process of gelation or setting is characterized by a discontinuous rise in viscosity. (See, P. I. Rose, “The Theory of the Photographic Process”, 4^{th} Edition, T. H. James ed. pages 51 to 67).

As used herein, the term “gelling agent” means a substance that can undergo gelation as described above. Examples include materials such as gelatin, water-soluble cellulose ethers or poly(n-isopropylacrylamide) that undergo thermal gelation or substances such as poly(vinyl alcohol) that may be chemically cross-linked by a borate compound. Other gelling agents may be polymers that may be cross-linked by radiation such as ultraviolet radiation or ionizing radiation or electron beam radiation. Examples of gelling agents include acacia, alginic acid, bentonite, carbomer, carboxymethylcellulose sodium, cetostearyl alcohol, colloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alcohol, povidone, propylene glycol alginate, sodium alginate, sodium starch glycolate, starch, tragacanth and xanthum gum. (For further discussion on gelling agents, see, accompanying reference Secundum Artem, Vol. 4, No. 5, Lloyd V. Allen). A preferred gelling agent is alkali pretreated gelatin.

As used herein, the term “random distribution” means a spatial distribution of elements showing no preference or bias. Randomness can be measured in terms of compliance with that which is expected by a Poisson distribution.

The present invention teaches a method for making a random array of microspheres, also referred to as “beads”, on a substrate, also called a receiving layer. The microspheres are deposited on the receiving layer in such a way that the surfaces of microspheres are exposed above the receiving layer. The distribution or pattern of the microspheres on the substrate is entirely random and the microspheres are not attracted or held to sites that are pre-marked or predetermined on the substrate as in other methods previously disclosed.

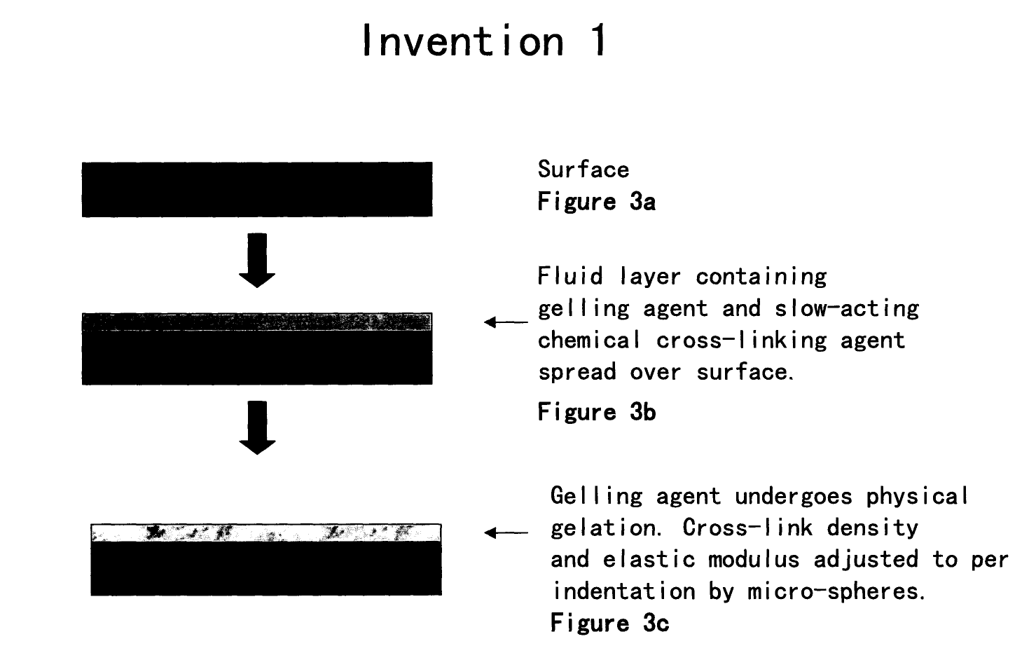

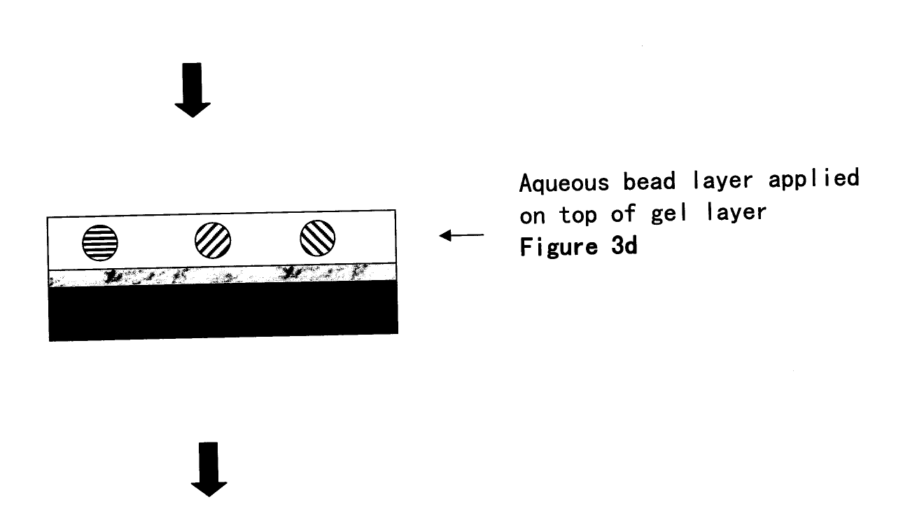

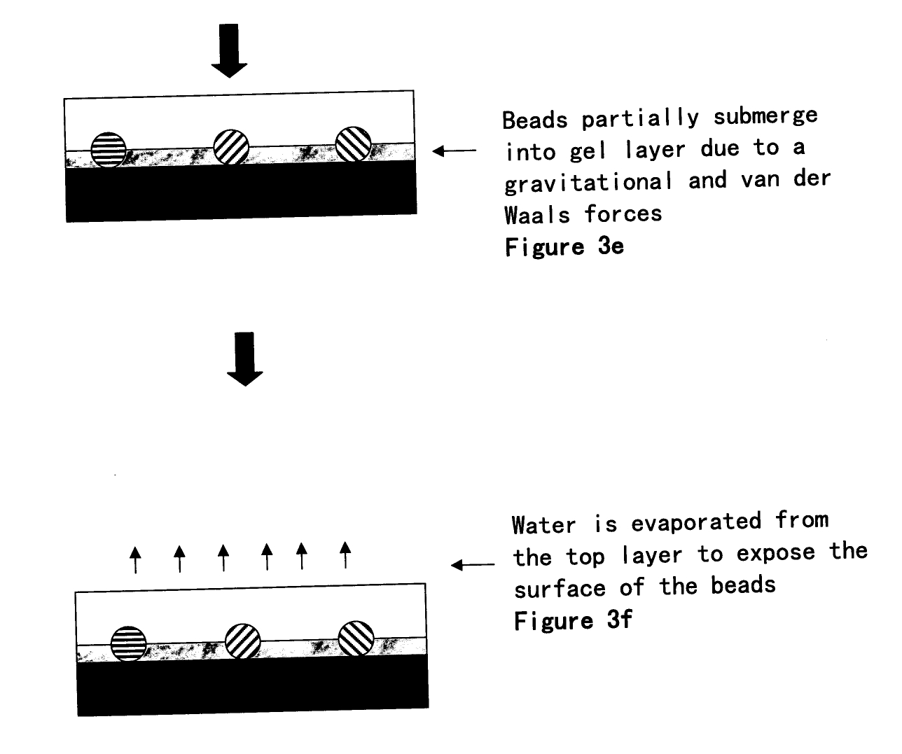

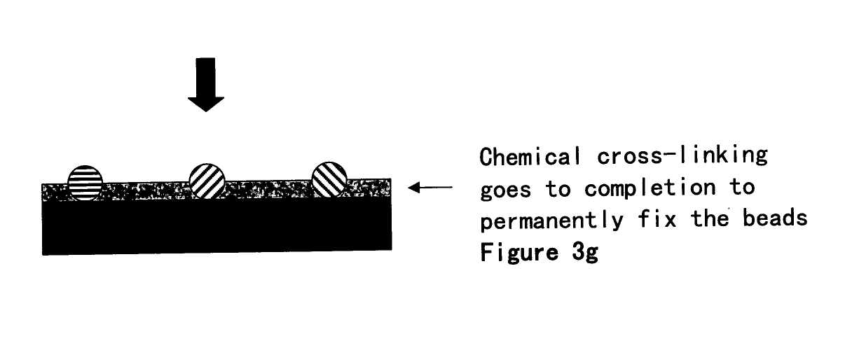

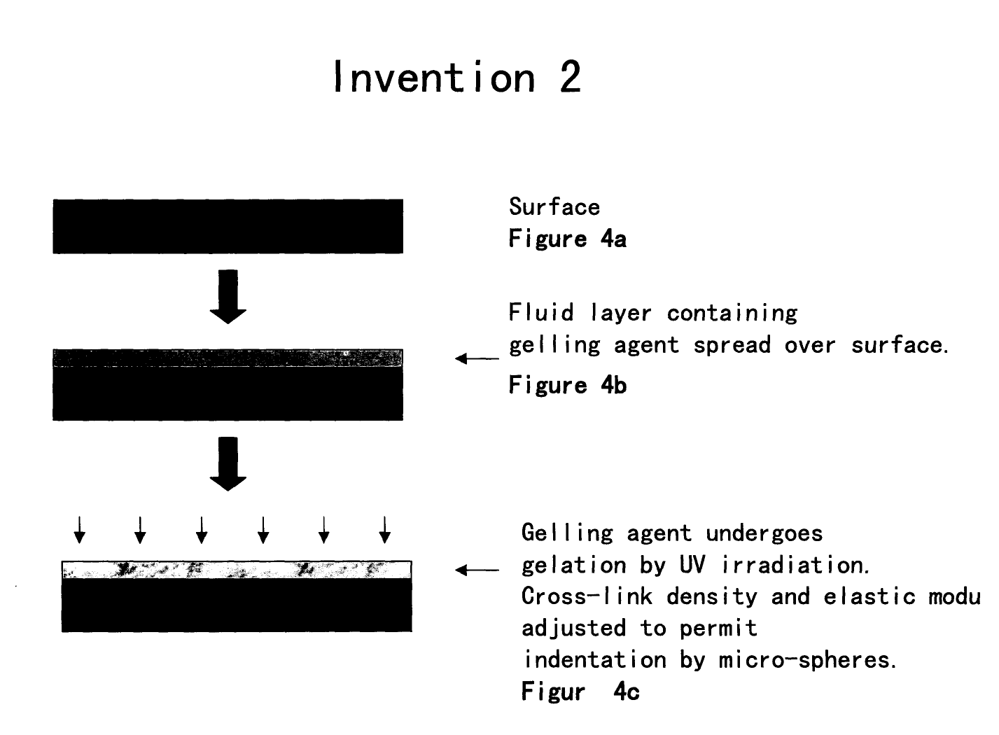

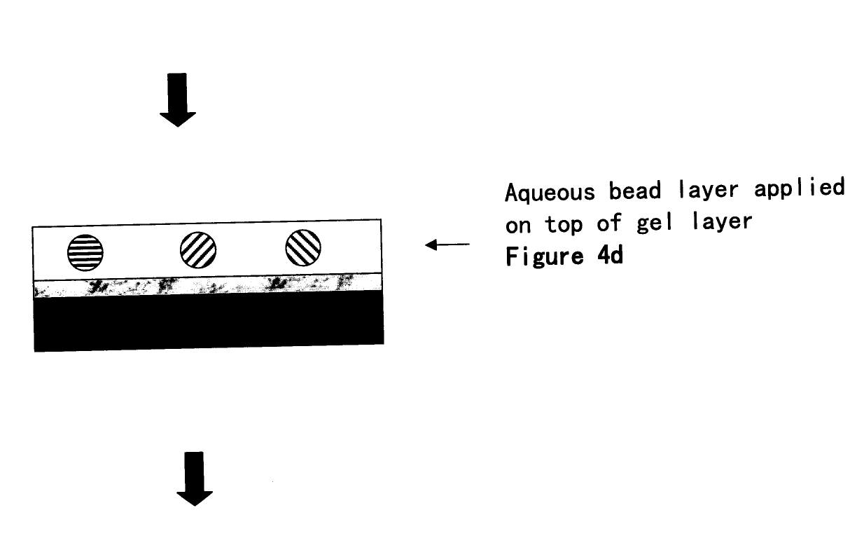

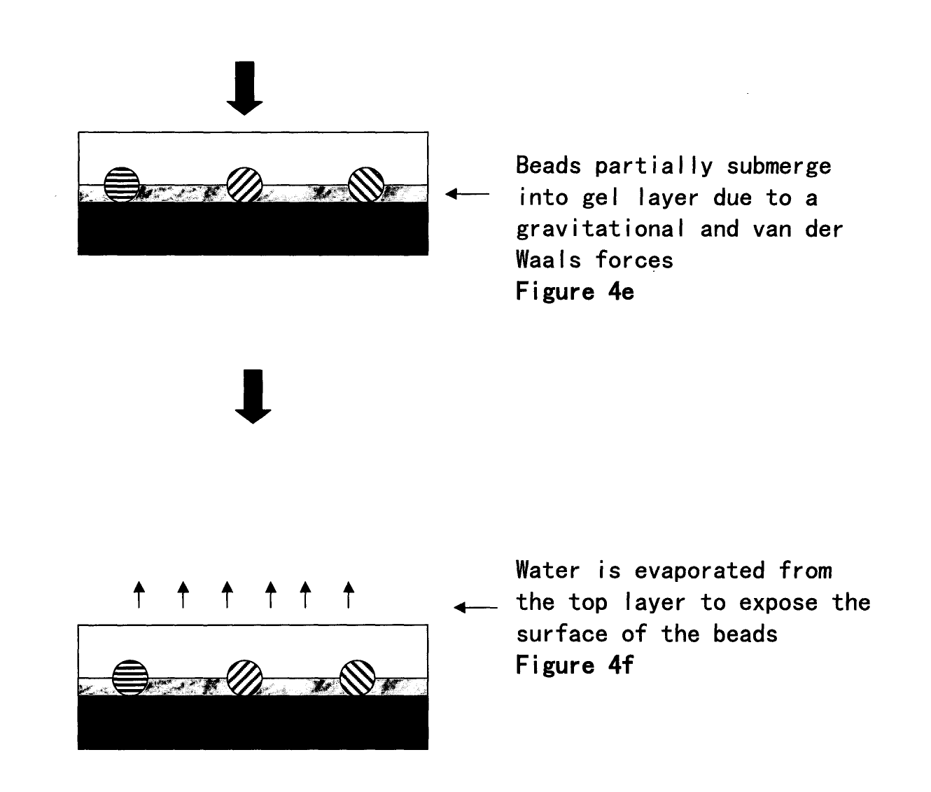

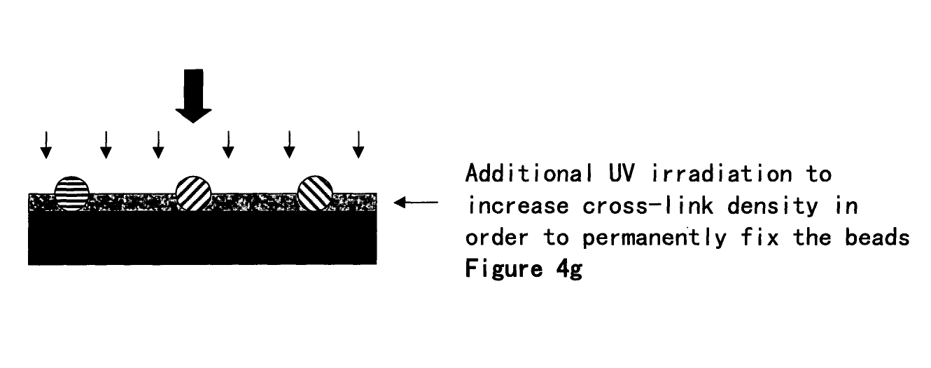

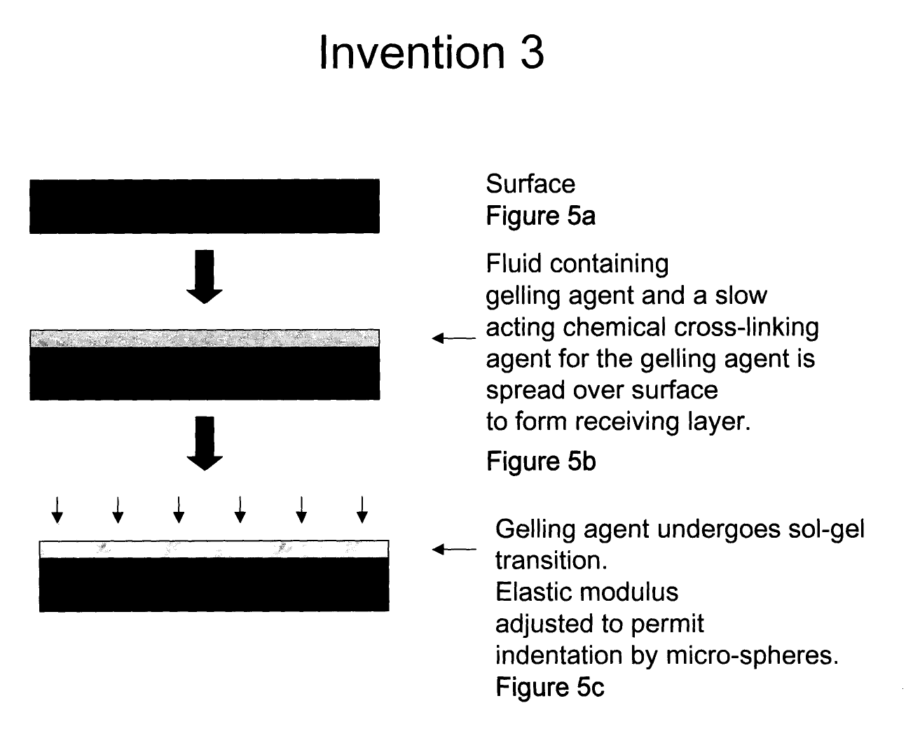

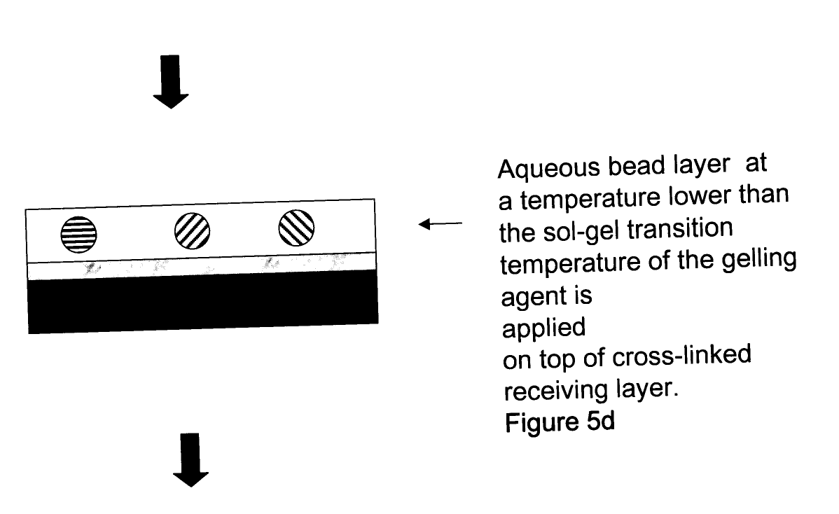

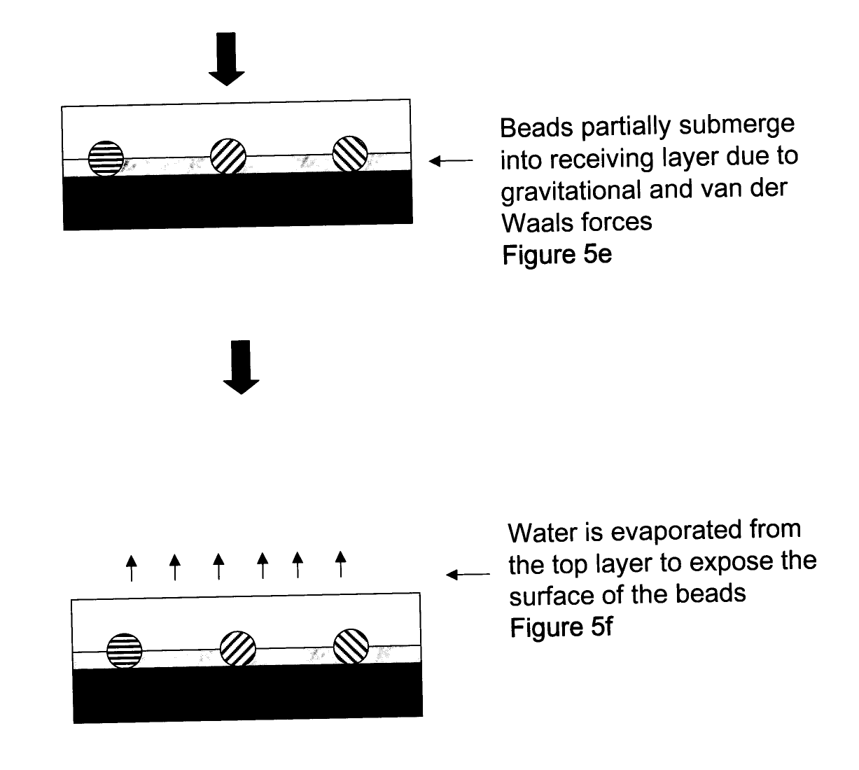

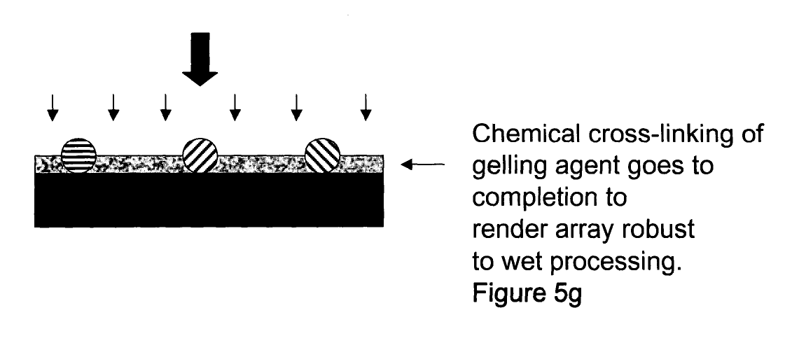

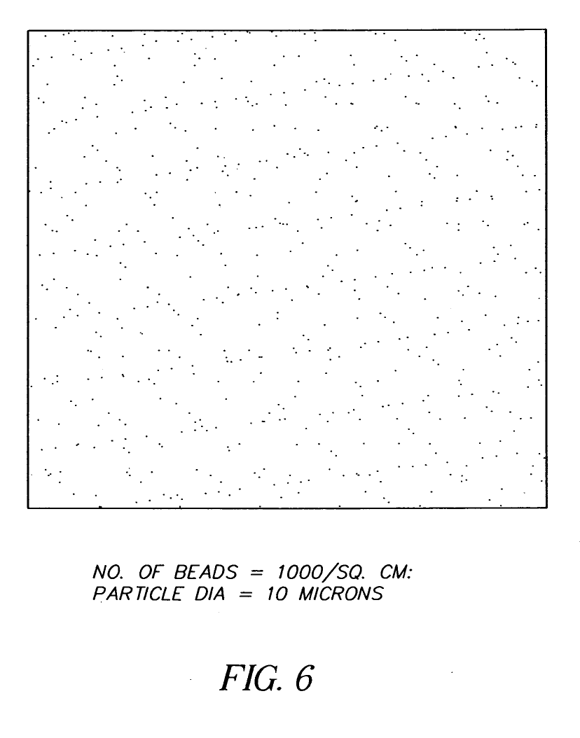

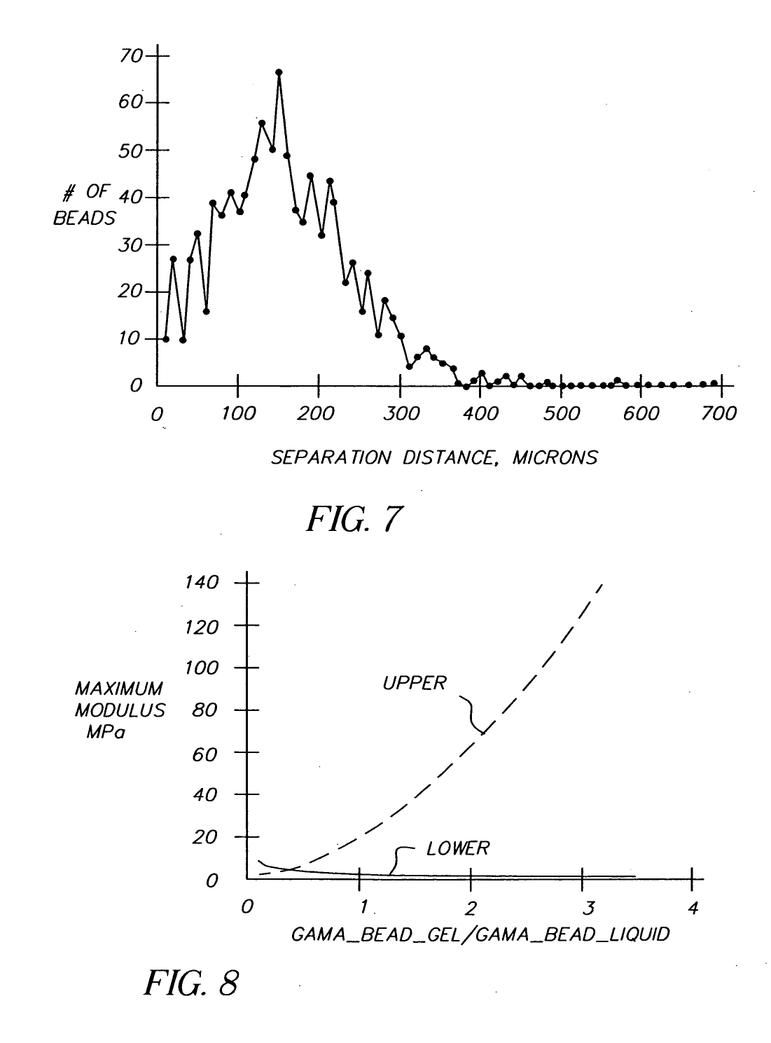

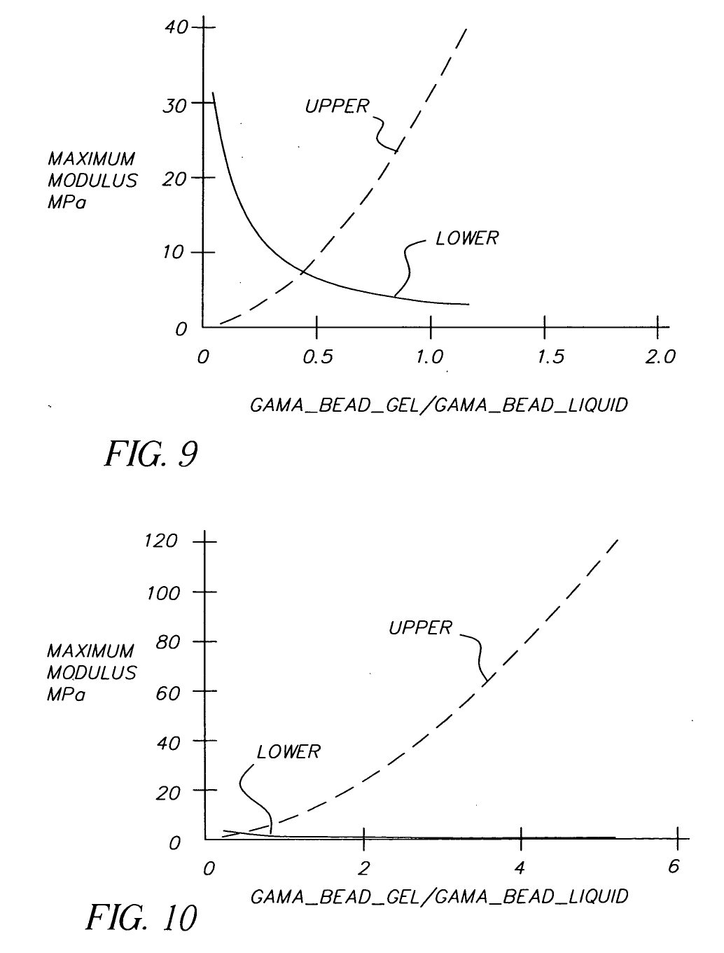

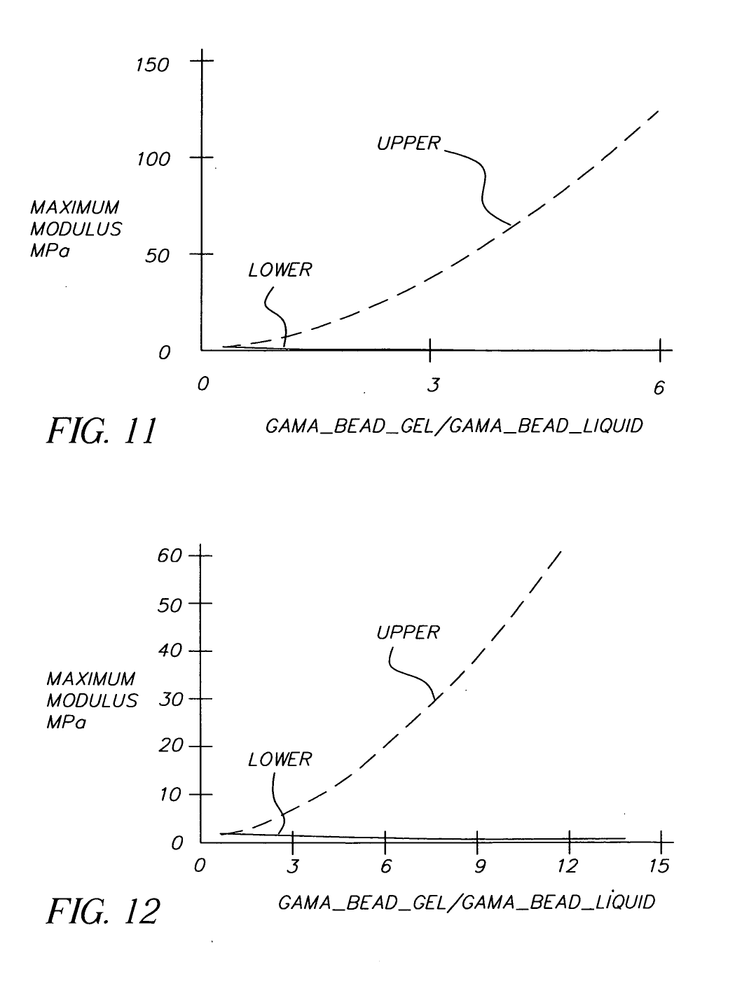

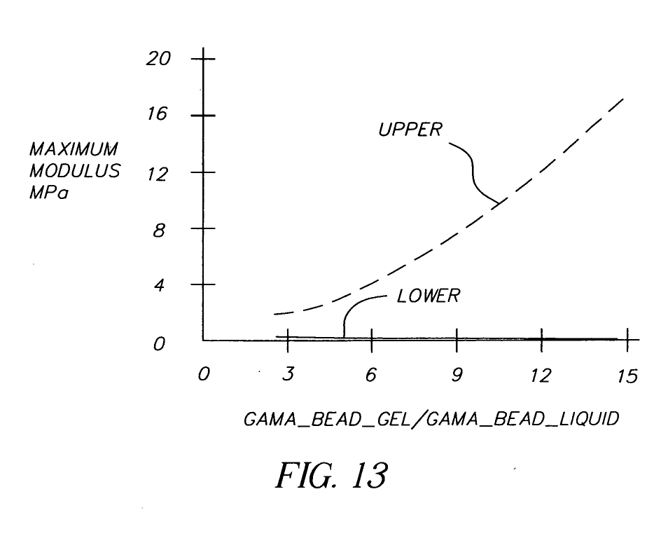

The random array is achieved by first coating on any suitable surface or support (FIGS. 3a, 4a, 5a) a fluid layer containing a gelling agent and a chemical cross-linker for the gelling agent. The support with the gelling agent is referred to as the receiving layer (FIGS. 3b, 4b, 5b). The gelling agent in the receiving layer is allowed to partially cross-link (FIGS. 3c, 4c, 5c). A fluid suspension of micro-spheres is then spread over the partially cross-linked receiving layer (FIGS. 3d, 4d, 5d). The cross-linked receiving layer is insoluble in the fluid medium. The micro-spheres settle into the cross-linked receiving layer (FIGS. 3e, 4e, 5e). The extent of settling is related to the elastic modulus of the receiving layer, the surface energy of the material of the micro-spheres and the surface energy of the receiving layer. The elastic modulus is controlled by the cross-link density defined as the moles of cross-links per unit volume. The cross-link density is in turn related to the concentration of chemical cross-linking agent, the duration of chemical cross-linking or the intensity and time of UV radiation or the dose rate and time of ionizing radiation depending on the type of cross-linking employed.

Alternatively, it is possible to use physical gelation instead of chemical cross-linking or radiation induced cross-linking. Physical gelation is based on formation of hydrogen bonds in the receiving layer. The cross-link density in physical gelation is related to the concentration of gelling agent and the difference between the temperature of the receiver and the gel point or sol-gel transition temperature of the gelling agent in the receiving layer. In this case, the temperature of the fluid suspension at the time of coating is maintained below the sol-gel transition temperature of the gelling agent in the receiving layer to prevent dissolution of the gelling agent in the receiving layer into the fluid suspension.

Evaporation of fluid from the array may be achieved by blowing air of a certain temperature and humidity over the array (FIGS. 3f, 4f, 5f). After the array has been fully fabricated on the coating device, the cross-linking reaction of the gelling agent initiated earlier by addition of the cross-linker may go to completion to permanently fix the micro-spheres in place. If gelatin is used as the gelling agent, preferred cross-linkers may be compounds such as bis(vinylsulfone)methane, glutaraldehyde or succinaldehyde. Alternatively, additional UV radiation, ionizing radiation or electron beam irradiation may be used to effect additional cross-linking.

The above procedure (physical gelation, chemical cross-linking or radiation before evaporation of fluid) is designed in a way that will yield a receiving layer with proper physical property to ensure that no lateral aggregation will occur during evaporation of fluid from the array. Two factors are important in determine if lateral aggregation will occurs. One is the capillary forces that drive the microspheres to each other (“Patterned Colloidal Deposition Controlled by Electrostatic and Capillary Forces”, J. Aizenberg, P. Braun, and P. Wiltzius, Physical Review Letters, Vol. 84, No. 13, 2000). The other is the degree of indentation of the microspheres into the receiving layer. (“Surface Energy and the Contact of Elastic Solids”, K. Johnson, K. Kendall, and A. Roberts, Proc. R. Soc. Lond, A. 324, 1971). The capillary force is proportional to the surface energy between the fluid and the microspheres. At the stage of fluid evaporation, when the thickness of the water layer become comparable to the microsphere size, the capillary force tend to cause lateral aggregation of microsphere. On the other hand, the surface force between the microspheres and the receiving layer (“Surface Energy and the Contact of Elastic Solids”, K. Johnson, K. Kendall, and A. Roberts, Proc. R. Soc. Lond, A. 324, 1971) causes the microsphere to indent into the relatively soft receiving layer. This will to permit sufficient submerging of the micro-spheres into the receiving layer to prevent lateral aggregation when the solvent in the suspension is removed by evaporation. The surface force is proportional to the surface energy between the microsphere and receiving layer.

From the above discussion, it is easy to see that to prevent lateral aggregation, the physical property of the receiving layer has to satisfy certain conditions. If the receiving layer is hard, there is very little submerging of the microsphere into the receiving layer, and the lateral aggregation is likely to occur. On the other hand, if the receiving layer is too soft, it will offer little resistance to the capillary force the drives lateral aggregation. If we use Young's modulus as a quantity to represent the material's property to resistant deformation there are a lower bound and an upper bound of the Young's modulus of the receiving layer that will ensure no lateral aggregation. Detailed information on the bounds of the Young's modulus of the receiving layer will be provided in the example section.

The invention discloses a polymeric latex bead based random microarray with each bead in the array having a distinct signature that would distinguish the bead. Such a signature may be based on color, shape or size of the bead. For signatures based on color, the color may be derived from mixing three dyes representing the primary colors R, G, B to create thousands of distinguishable beads with distinct “color addresses” (unique RGB values, e.g. R=0, G=204, B=153). The beads can be made with sites on their surface that are “active”, meaning that at such sites physical or chemical interaction can occur between the bead and other molecules or compounds. Such compounds may be organic or inorganic. Usually, the molecule or compound is organic—nucleic acid, protein or fragments thereof, are examples. To the surface of each color coded bead may be attached a pre-synthesized oligonucleotide, a monoclonal antibody, or any other biological agents. Therefore, each color address can correspond to a specific bioactive probe. These beads may be mixed in equal amounts, and the random microarray fabricated by coating the mixed beads in a single or multilayer format.

Coating methods are broadly described by Edward Cohen and Edgar B. Gutoff in Chapter 1 of “Modern Coating And Drying Technology”, (Interfacial Engineering Series; v.1), (1992), VCH Publishers Inc., New York, N.Y. Suitable coating methods may include knife coating and blade coating.

Fluorescently/chemiluminescently labeled biological sample can be hybridized to the bead based random microarray. The signals from both “color addressable” polymeric beads and biological sample non-selectively labeled with fluorescence/chemiluminescence may be analyzed by a charge coupled device after image enlargement through an optical system. The recorded array image can be automatically analyzed by an image processing algorithm to obtain bioactive probe information based on the RGB color code of each bead, and the information compared to the fluorescence/chemiluminescence image to detect and quantify specific biological analyte materials in the sample. Optical or other electro-magnetic means may be applied to ascertain signature.

Although microspheres or particles having a substantially curvilinear shape are preferred because of ease of preparation, particles of other shape such as ellipsoidal or cubic particles may also be employed. Suitable methods for preparing the particles are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used. The formed microsphere is colored using an insoluble colorant that is a pigment or dye that is not dissolved during coating or subsequent treatment. Suitable dyes may be oil-soluble in nature. It is preferred that the dyes are non-fluorescent when incorporated in the microspheres.

The microspheres are desirably formed to have a mean diameter in the range of 1 to 50 microns; more preferably in the range of 3 to 30 microns and most preferably in the range of 5 to 20 microns. It is preferred that the concentration of microspheres in the coating is in the range of 100 to a million per cm², more preferably 1000 to 200,000 per cm² and most preferably 10,000 to 100,000 per cm².

The attachment of bioactive agents to the surface of chemically functionalized microspheres can be performed according to the published procedures in the art (Bangs Laboratories, Inc, Technote #205). Some commonly used chemical functional groups include, but not limited to, carboxyl, amino, hydroxyl, hydrazide, amide, chloromethyl, epoxy, aldehyde, etc. Examples of bioactive agents include, but are not limited to, oligonucleotides, DNA and DNA fragments, PNAs, peptides, antibodies, enzymes, proteins, and synthetic molecules having biological activities.

### EXAMPLES

In the following example, Monte Carlo simulations are performed to determine the distance between the microspheres where introduced randomly. The results are then utilized in the analysis that leads to the lower and upper bounds of the Young's modulus of the receiving layer that will avoid lateral aggregation of microspheres.

In FIG. 6, 1000 beads (of 10μ diameter) were randomly dropped over an area of 1 cm², such that no two of them overlap. Table 1 shows the distribution of nearest neighbor separation distances between the beads, and FIG. 7 is a plot of the data in Table 1. The simulation in FIG. 6 was repeated 20 times, and the average over all simulations is represented in Table 2.

Column 3 in Table 2 indicates that for this particular example (1000 beads/sq.cm; 10μ diameter beads), 95% of the beads are separated from their nearest neighbors by more than 30μ. 30μ is thus determined as “L” for this example.

The example was repeated for several cases of bead density and bead diameter, and “L” was determined as described above. The results are summarized in Table 3.

Using the values from Table 3, one can proceed to determine the modulus requirement for the gel layer to anchor the micro-beads without lateral aggregation. As shown in FIG. 8 (number of beads/cm\hat{ }2=1000, bead diameter=10μ, L=30μ), the feasible modulus is determined from the lower curve (lower bond) and upper curve (upper bond). Of course, the result depends on the magnitude of the anchoring force (represented by the surface energy between the microsphere and the receiving layer, gama_bead_gel) and the lateral force (represented by the surface energy between the microsphere and the fluid, gama_bead_liquid). For instance, when the ratio of anchoring force to the lateral force is equal to 2, the modulus of the gel layer should be between 1 MPa to 55 MPa, as shown in FIG. 8. The results for other cases from Table 3 are shown in FIGS. 9-13. The ratio between the anchoring force and lateral force is normally between 1 to 4. For practical purposes, the lower bound for the modulus can be chosen as 1 MPa, while the upper bound is over 100 MPa, depending on the number of bead per unit area, the bead radius and bead separation distance L, as shown in FIGS. 8-13.

Table 1 shows the distribution of nearest neighbor separation distances between the beads, and FIG. 7 is a plot of the data in Table 1.

TABLE 1

| Nearest neighbor | |
|---|---|
| separation distance, μ | No. of beads |
| 0-10 | 10 |
| 10-20 | 27 |
| 20-30 | 10 |
| 30-40 | 27 |
| 40-50 | 32 |
| 50-60 | 16 |
| 60-70 | 38 |
| 70-80 | 36 |
| 80-90 | 41 |
| 90-100 | 37 |
| 100-110 | 40 |
| 110-120 | 48 |
| 120-130 | 55 |
| 130-140 | 50 |
| 140-150 | 66 |
| 150-160 | 49 |
| 160-170 | 37 |
| 170-180 | 35 |
| 180-190 | 44 |
| 190-200 | 32 |
| 200-210 | 43 |
| 210-220 | 39 |
| 220-230 | 22 |
| 230-240 | 26 |
| 240-250 | 16 |
| 250-260 | 24 |
| 260-270 | 11 |
| 270-280 | 18 |
| 280-290 | 15 |
| 290-300 | 11 |
| 300-310 | 4 |
| 310-320 | 6 |
| 320-330 | 8 |
| 330-340 | 6 |
| 340-350 | 5 |
| 350-360 | 4 |
| 360-370 | 1 |
| 370-380 | 0 |
| 380-390 | 1 |
| 390-400 | 3 |
| 400-410 | 0 |
| 410-420 | 1 |
| 420-430 | 2 |
| 430-440 | 0 |
| 440-450 | 2 |
| 450-460 | 0 |
| 460-470 | 0 |
| 470-480 | 1 |
| 480-490 | 0 |
| 490-500 | 0 |
| 500-510 | 0 |
| 510-520 | 0 |
| 520-530 | 0 |
| 530-540 | 0 |
| 540-550 | 0 |
| 550-560 | 0 |
| 560-570 | 1 |
| 570-580 | 0 |
| 580-590 | 0 |
| 590-600 | 0 |
| 600-610 | 0 |
| 610-620 | 0 |
| 620-630 | 0 |
| 630-640 | 0 |
| 640-650 | 0 |
| 650-660 | 0 |
| 660-670 | 0 |
| 670-680 | 0 |
| 680-690 | 0 |
| 690-700 | 0 |
| Total: | 1000 |

The simulation in FIG. 6 was repeated 20 times, and the average over all simulations is represented in Table 2. Column 3 in Table 2 indicates that for this particular example (1000 beads/sq.cm; 10μ diameter beads), 95% of the beads are separated from their nearest neighbors by more than 30μ. 30μ is thus determined as “L” for this example.

TABLE 2

| Nearest neighbor | No. of beads (ave. | Cumulative average (% |
|---|---|---|
| separation distance, μ | over 20 simulations) | of total # of beads) |
| 0-10 | 9.4 | 100 |
| 10-20 | 16.65 | 99.06 |
| 20-30 | 22.4 | 97.395 |
| 30-40 | 25.3 | 95.155 |
| 40-50 | 32.9 | 92.625 |
| 50-60 | 34.6 | 89.335 |
| 60-70 | 37.4 | 85.875 |
| 70-80 | 39.55 | 82.135 |
| 80-90 | 42.55 | 78.18 |
| 90-100 | 42.05 | 73.925 |
| 100-110 | 45.8 | 69.72 |
| 110-120 | 47.5 | 65.14 |
| 120-130 | 49.3 | 60.39 |
| 130-140 | 52.2 | 55.46 |
| 140-150 | 45.7 | 50.24 |
| 150-160 | 47 | 45.67 |
| 160-170 | 40.15 | 40.97 |
| 170-180 | 39.15 | 36.955 |
| 180-190 | 37.2 | 33.04 |
| 190-200 | 32.4 | 29.32 |
| 200-210 | 31.5 | 26.08 |
| 210-220 | 28.05 | 22.93 |
| 220-230 | 28.65 | 20.125 |
| 230-240 | 25.95 | 17.26 |
| 240-250 | 21.5 | 14.665 |
| 250-260 | 17.85 | 12.515 |
| 260-270 | 14.8 | 10.73 |
| 270-280 | 16.1 | 9.25 |
| 280-290 | 11.35 | 7.64 |
| 290-300 | 9.75 | 6.505 |
| 300-310 | 7.8 | 5.53 |
| 310-320 | 8.85 | 4.75 |
| 320-330 | 6.75 | 3.865 |
| 330-340 | 6.1 | 3.19 |
| 340-350 | 4.35 | 2.58 |
| 350-360 | 3.4 | 2.145 |
| 360-370 | 2.55 | 1.805 |
| 370-380 | 2.8 | 1.55 |
| 380-390 | 2.6 | 1.27 |
| 390-400 | 1.45 | 1.01 |
| 400-410 | 1.8 | 0.865 |
| 410-420 | 1.15 | 0.685 |
| 420-430 | 1.45 | 0.57 |
| 430-440 | 0.9 | 0.425 |
| 440-450 | 0.55 | 0.335 |
| 450-460 | 0.7 | 0.28 |
| 460-470 | 0.45 | 0.21 |
| 470-480 | 0.6 | 0.165 |
| 480-490 | 0.3 | 0.105 |
| 490-500 | 0.1 | 0.075 |
| 500-510 | 0.2 | 0.065 |
| 510-520 | 0.1 | 0.045 |
| 520-530 | 0.05 | 0.035 |
| 530-540 | 0 | 0.03 |
| 540-550 | 0 | 0.03 |
| 550-560 | 0.05 | 0.03 |
| 560-570 | 0.05 | 0.025 |
| 570-580 | 0 | 0.02 |
| 580-590 | 0.05 | 0.02 |
| 590-600 | 0 | 0.015 |
| 600-610 | 0.05 | 0.015 |
| 610-620 | 0.05 | 0.01 |
| 620-630 | 0 | 0.005 |
| 630-640 | 0 | 0.005 |
| 640-650 | 0 | 0.005 |
| 650-660 | 0 | 0.005 |
| 660-670 | 0 | 0.005 |
| 670-680 | 0.05 | 0.005 |
| 680-690 | 0 | 0 |
| 690-700 | 0 | 0 |

The example was repeated for several cases of bead density and bead diameter, and “L” was determined as described above. The results are summarized in Table 3.

TABLE 3

| # of beads/cm² | bead dia, μ | L, μ |
|---|---|---|
| 1000 | 5 | 30* |
| 1000 | 10 | 30** |
| 1000 | 15 | 20* |
| 1000 | 20 | 20* |
| 10000 | 10 | 5* |
| 10000 | 20 | 2.5* |

*96% of beads are separated by >L from their nearest neighbors
**95% of beads are separated by >L from their nearest neighbors

1. A method for fabricating an element containing an array of microspheres on a support, the method comprising the steps of:
a) coating a support with a coating composition to form an intermediate receiving layer, said layer having a modulus that can be modified by crosslinking;
b) allowing partial cross-linking to proceed in the intermediate layer to achieve an elastic modulus that permits partial submerging of the micro-spheres into the receiving layer;
c) coating on the partially cross-linked receiving layer a dispersion of microspheres in a carrier fluid;
d) allowing the microspheres to partially submerge into the intermediate layer;
e) removing the fluid medium from the suspension of microspheres; and
e) fixing the microspheres on the intermediate layer so that the element can withstand wet processing.

2. The method of claim 1 wherein said microspheres form an interface with the intermediate layer, said interface having an interfacial energy of γ₁.

3. The method of claim 1 wherein said microspheres form an interface with the fluid medium, said interface having an interfacial energy of γ₂.

4. The method of claim 1 wherein the modulus of said intermediate layer has a lower bound of 1 MPa.

5. The method of claim 1 wherein the modulus of said intermediate layer has an upper bound defined by a monotonically increasing function of the ratio of γ₁ to γ₂.

6. The method of claim 5 wherein slope of said function varies depending on number of beads per unit area and bead diameter.

7. The method of claim 5 wherein the upper bound is 19 MPa when ratio of γ₁ to γ₂ is 1 and the number of beads per cm² is 1000 and the bead diameter is 10μ.

8. The method of claim 5 wherein the upper bound is 63 MPa when ratio of γ₁ to γ₂ is 2 and the number of beads per cm² is 1000 and the bead diameter is 10μ.

9. The method of claim 5 wherein the upper bound is 128 MPa when ratio of γ₁ to γ₂ is 3 and the number of beads per cm² is 1000 and the bead diameter is 10μ.

10. The method of claim 5 wherein the upper bound is 31 MPa when ratio of γ₁ to γ₂ is 1 and the number of beads per cm² is 1000 and the bead diameter is 5μ.

11. The method of claim 5 wherein the upper bound is 22 MPa when ratio of γ₁ to γ₂ is 2 and the number of beads per cm² is 1000 and the bead diameter is 15μ.

12. The method of claim 5 wherein the upper bound is 75 MPa when ratio of γ₁ to γ₂ is 4 and the number of beads per cm² is 1000 and the bead diameter is 15μ.

13. The method of claim 5 wherein the upper bound is 10 MPa when ratio of γ₁ to γ₂ is 1 and the number of beads per cm² is 1000 and the bead diameter is 20μ.

14. The method of claim 5 wherein the upper bound is 40 MPa when ratio of γ₁ to γ₂ is 3 and the number of beads per cm² is 1000 and the bead diameter is 20μ.

15. The method of claim 5 wherein the upper bound is 125 MPa when ratio of γ₁ to γ₂ is 6 and the number of beads per cm² is 1000 and the bead diameter is 20μ.

16. The method of claim 5 wherein the upper bound is 7 MPa when ratio of γ₁ to γ₂ is 3 and the number of beads per cm² is 10000 and the bead diameter is 10μ.

17. The method of claim 5 wherein the upper bound is 20 MPa when ratio of γ₁ to γ₂ is 6 and the number of beads per cm² is 10000 and the bead diameter is 10μ.

18. The method of claim 5 wherein the upper bound is 38 MPa when ratio of γ₁ to γ₂ is 9 and the number of beads per cm² is 10000 and the bead diameter is 10μ.

19. The method of claim 5 wherein the upper bound is 2 MPa when ratio of γ₁ to γ₂ is 3 and the number of beads per cm² is 10000 and the bead diameter is 20μ.

20. The method of claim 5 wherein the upper bound is 4 MPa when ratio of γ₁ to γ₂ is 6 and the number of beads per cm² is 10000 and the bead diameter is 20μ.

21. The method of claim 5 wherein the upper bound is 7 MPa when ratio of γ₁ to γ₂ is 9 and the number of beads per cm² is 10000 and the bead diameter is 20μ.

22. An element comprising randomly fixed microspheres, the element comprising:
a) a support; on which is disposed
b) a water-insoluble, crosslinked layer; and
(c) microspheres partially submerged in the crosslinked layer;
wherein the microspheres, upon deposition on the layer, have surfaces exposed above the crosslinked layer, such exposed surfaces having probes attached that are capable of interacting with analytes applied to the element.

23. The element of claim 22 wherein the crosslinkable layer contains gelatin.

24. The element of claim 22 wherein the microspheres comprise polystyrene or polymethylmethacrylate.

25. The element of claim 22 wherein the support comprises glass paper, metal or polymer.

26. The element of claim 22 wherein the microspheres have a diameter of 1 to 100μ.

27. The element of claim 22 wherein the microspheres have a diameter of 5 to 20μ.

28. The element of claim 22 wherein the number of micropheres per cm² is between 1 and 1,000,000.

29. The element of claim 22 wherein the number of micropheres per cm² is between 100 and 100,000.

30. The element of claim 22 wherein the number of micropheres per cm² is between 1000 and 10,000.

31. The element of claim 22 wherein the microspheres are color coded.

32. The element of claim 31 wherein the color code identifies the probe on the surface of the microsphere.

33. The element of claim 22 wherein the probe is protein or nucleic acid.

34. An element produced by a method comprising the steps of:
a) coating a support with a coating composition to form an intermediate receiving layer, said layer having a modulus that can be modified by crosslinking;
b) allowing partial cross-linking to proceed in the intermediate layer to achieve an elastic modulus that permits partial submerging of the micro-spheres into the receiving layer;
c) coating on the partially cross-linked receiving layer a dispersion of microspheres in a carrier fluid;
d) allowing the microspheres to partially submerge into the intermediate layer;
e) removing the fluid medium from the suspension of microspheres; and
f) fixing the microspheres on the intermediate layer so that the element can withstand wet processing.
